(19) United States
(12) Patent Application Publication
(10) Pub. No.: US 2012/0108899 A1
(43) Pub. Date: May 3, 2012

(54) DEVICE FOR THE CONTROLLED INFUSION OF LIQUID FORMULATIONS IN TISSUES AND ORGANS IN CELLULAR THERAPEUTIC PROCEDURES

(76) Inventors: Luis Eduardo Da Cruz, Rio de Janerio (BR); Enio Buffolo, Sao Paulo (BR); Nelson Americo Hossne Júnior, Sao Paulo (BR); Adriana Luckow Invitti, Sao Paulo (BR)

(21) Appl. No.: 13/258,423

(22) PCT Filed: Mar. 17, 2010
(86) PCT No.: PCT/BR10/00092

§ 371 Date: Jan. 12, 2012

Related U.S. Application Data
(60) Provisional application No. 61/161,037, filed on Mar. 17, 2009.

(30) Foreign Application Priority Data
Jun. 16, 2009 (BR) PI0901908-1

(51) Int. Cl. A61M 5/46; A61B 1/018; A61B 1/04; A61M 5/178

(52) U.S. Cl. 600/106; 604/117; 604/187

(57) ABSTRACT

The present invention relates to a device for the controlled infusion of liquid formulations, especially formulations comprising cells, directly into tissues or organs, to be used in cellular therapeutic procedures related to ischemic or non-ischemic diseases. The device comprises an automatic dispenser (1) connected to a reservoir (2), which is connected to a duct (3). The duct (3) comprises in its opposite end a system consisting of a grip wing (4) connected to a needle (6) comprising a depth insertion control device (5).

### FIELD OF THE INVENTION

The present invention relates to a device for the controlled infusion of liquid formulations, especially formulations comprising cells, directly into tissues or organs, to be used in cellular therapeutic procedures related to ischemic or non-ischemic diseases or lesions of the heart, brain, upper and lower limbs, spinal cord or any other organ or tissue.

### BACKGROUND OF THE INVENTION

The researches using stem cells have increased a lot in the last years. The cellular therapy is no longer an exclusive treatment for hematologic diseases. Nowadays, several diseases, mainly of ischemic etiology, are being treated with stem cells (stroke, coronary disease, among others [Tse, H-F. et al. (2007); Steinhoff, G. et al. (2006); Ting, A. E. et al. (2008); Guan, K. e Hasenfuss, G. (2007); Aversa, P. et al. (2007); Haider, H. Kh. e Ashraf, M. (2005); Laflamme, M. A. (2007); Losordo, D. W. e Renault, M. A. (2007); Garbuzova-Davis, S. et al. (2002); Garbuzova-Davis, S. et al. (2003); Saporta, S. et al. (2003); Garbuzova-Davis, S. et al. (2005); Garbuzova-Davis, S. et al. (2006); Chen, J. et al. (2001); Sanchez-Ramos, J. R. et al. (2001); Zigova, T. et al. (2002); Willing, A. E. et al. (2003); Henning, R. J. et al. (2004); Vendrame, M. et al. (2004); Garbuzova-Davis, S. et al. (2005); Newman, M. B. et al. (2005); Vendrame, M. et al. (2005); Newcomb, J. D. et al. (2006); Newman, M. B. et al. (2006); Vendrame, M. et al. (2006); Li, Y. e Chopp, M. (2009)].

The significant growth of the cellular therapy allowed the identification of a need for development of medical products (devices—systems and products) to make easier the administration of cellular solutions, mainly for Cardiac Cellular Therapy, to facilitate the procedure and make it safer and widely available.

The cellular therapy, for at least 10 years, has been studied as an alternative for treatment of ischemic etiology diseases. In the cardiology area, the results obtained in clinical trials in the last years are promising [Orlic D. et al. (2001); Lunde, K. et al. (2006); Schachinger, V. et al. (2004); Schachinger, V. et al. (2006); Schachinger, V. et al. (2006); Chen , S. L. (2004); Meyer, G. P. et al. (2006); Janssens, S. et al. (2006); Strauer, B. E. et al. (2002); Bartunek, J. et al. (2005); Kang, H. J. et al. (2006); Li, Z. A. et al. (2007); Assmus, B. et al. (2002); Ge, J. et al. (2006); Meluzin, J. et al. (2006); Hossne et al. (2009)] and enable the transfer of this kind of procedure for medical practice. Considering only a subgroup of the ischemic cardiovascular disease, chronic coronary disease, it is estimated that about 100,000 patients per year in Brazil could benefit from the cellular therapy [Ministry of Health, Brazilian Government (2005)].

The equipments and devices for cellular therapy in ischemic cardiovascular disease are very recent and most are still in testing phase. Modern equipments still have several limitations in the use of medical practice. For instance, the electromechanical mapping system and cells infusion (NOGA®—U.S. Pat. No. 5,738,096 and patent application US 2007/0059288) that performs the intra-myocardial infusion via catheter [Beeres, SLMA et al. (2006); Losordo, D. W. et al. (2007); Van Ramshorst, J. et al. (2009); Perin, E. C. et al. (2003); Fuchs, S. et al. (2003); Fuchs, S. et al. (2006); Tse, H-F. et al. (2006); Tse, H-F. et al. (2003); Tse, H-F. et al. (2007)], presenting physical limitations of access to ischemic areas due to the caliber and the ability to flex the catheter, aortic diameter and anatomy of the left ventricle. These factors reduce the infusion accuracy and limit the access of some left ventricle areas. In addition, the endomyocardium is trabecular difficulting the intramyocardium infusion, since the needle could be introduced in the intermuscular spaces and the cells released into the blood.

The stem cells infusion in tissues and organs also could be performed by syringes. In this case, the cellular suspension would be longer in contact with the air, until all the syringes needed for the procedure are fulfilled. Furthermore, it is not possible to precise the injected volume (parallax effect) and the needle insertion depth and liquid infusion speed may vary according the manipulator [Henring, R. J. et al. (2007)].

Within this context, the present invention relates to an infusion device of liquid formulations for cellular therapy.

### SUMMARY OF THE INVENTION

The infusion device of the present invention aims to facilitate and standardize the cellular therapy, ensuring the accuracy and reproducibility of the cellular suspension infusion or any kind of liquid comprising cells (suspended cells in suitable liquids to the proposed procedure, as solutions comprising different salt concentrations, biological molecules, different cell types, culture media, etc.) in predetermined volumes, speeds, and injection depths, directly in desired locations into tissues or organs in clinical, surgical or minimally invasive cellular therapy procedures.

The infusion device of the present invention may comply with the Therapeutic Good Clinical Practice protocol, and then, to assure the reproducibility in the conversion of the pre-clinical and clinical data and results, in an accurate and controlled way to patients once it is approved. The infusion device of the present invention is unique as it helps cellular and regenerative scientific medicine and clinical trials to accurately adhere to GMP (Good Manufacturing Practices) and GCP (Good Clinical Practices).

The use of the device of the present invention assures better results in clinical outcomes and benefits to the treated patients. This system permits an exact dosage determination, and reproducibility of this dosage during the treatment.

The infusion device of the present invention comprises an automatic dispenser connected to a reservoir, which contains the cellular suspension or any kind of liquid comprising cells to be infused. One end of the reservoir, preferably its bottom, is connected to a duct, preferably made of vinyl, which presents in the opposite end a system consisting of a grip wing/handle connected to a needle comprising a depth insertion control device.

The duct presence allows greater flexibility and mobility to the system, which become a huge advantage when the system is used in surgical procedures; to allow the infusion in difficult access areas. The duct absence makes the things easier in body surface procedures, where the stability of the system is more important than its malleability. This variation makes the infusion system described here be effective in both clinical and surgical procedures, with minimal variations in their presentation.

### BRIEF DESCRIPTION OF THE DRAWINGS

The structure and operation of the present invention, as well as its advantages may be better understood by the attached drawings, in which:

FIG. 1: represents a perspective view of an infusion device according to the present invention;

FIG. 2: represents a perspective view of an infusion device according the present invention, showing the automatic dispenser connected with a reservoir; and

FIG. 3: represents a perspective view of an infusion device according the present invention, showing the reservoir and the duct having a system consisting of a grip wing/handle connected to a needle comprising a depth insertion control device.

### DETAILED DESCRIPTION OF THE INVENTION

Although the present invention may encompass different embodiments, preferred embodiments of the invention are described in the specification and drawings. Such preferred embodiments illustrated and described herein do not limit the scope of the present invention.

In prior art systems, the infusion process is carried out manually, using several apparatus, from different sources and types, and the accuracy of the infused volume, speed and depth of injection may easily vary between different users. This represents risks and often leads to unsuccessful or inconsistent pre-clinical, clinical or therapeutic results, as well as in discordance with the results of clinical trials or previous procedures. In compliance with previous clinical trial results or proceedings, it is a very important issue for a scientific laboratory finding to be converted to the clinical setting. In addition, proceeding is tried in a number of individuals larger than the test group; even in the test group, it is difficult to evaluate whether or not there have been variations in speed, location, amount of cells or liquid volume and depth of the needle injection, which also represents risk.

Thus, by providing the controlled infusion of pre-determined volumes at pre-determined speed and depth, the present invention assures reproducibility of clinically proven mean volumes, speed and depth.

This reproducibility property of the present invention related with the dispensed infusion volume, speed and depth, ensures the accuracy and specificity of liquid perfusion; increasing process accuracy and compliance with the GCP.

Moreover, the infusion device of the present invention ensures the accuracy and reproducibility of the clinical therapeutic results, by providing the infusion of cellular suspensions or any kind of liquid comprising cells in pre-determined volumes, speeds, and injection depths, directly in desired, or pre-determined locations clinically proven, in tissues or organs affected by ischemic or non-ischemic diseases, in clinical, surgical or minimally invasive medical procedures. The reproducibility of the processes allows a short-time spent in clinical-surgical procedures when compared to the usual clinical practice. The time spent in the infusion procedure is significantly reduced, consequently the risks are reduced and the clinical benefits are increased.

Furthermore, the infusion device of the present invention also allows the procedure be performed with minimum contact of the liquid to be infused with the environment, thus reducing the risk of microbiologic contamination.

In addition, in another embodiment of the present invention, the infusion device may comprises the addition of an imaging system apparatus that allows an even better accuracy and control, by providing the user with a series of images of the organ or tissue, in which the formulation will be infused, during the pre-infused, per-infused and post-infused. The imaging device comprises, preferably, a micro-camera coupled to the infusion device, preferably, in the grip wing/handle.

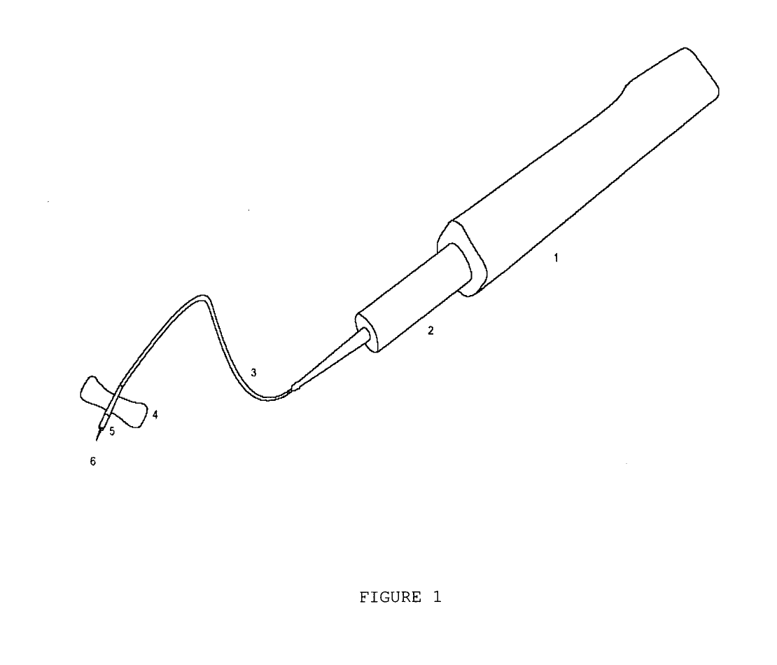

FIG. 1 shows an embodiment of the infusion device of the present invention. According to the preferred embodiment of the present invention, it comprises an automatic dispenser (1), an automatic pipette which provides an automatic suction and dispensing control of the liquid to be infused. The automatic dispenser is connected to a reservoir (2), preferably made of plastic, preferably fully sterile, which contains the liquid to be infused. One end of the reservoir, preferably its bottom (2) is connected to a duct (3) or directly to a needle. The duct is preferably made of transparent latex-free PVC with 5 to 50 cm of length, which comprises in the opposite end a system consisting of a grip wing/handle (4) connected to a needle (6) preferably having a caliber between 19-27 gauges (0.91 mm to 0.36 mm), comprising a depth insertion control device (5), which limits the insertion in 0, 1 to 1 cm depth.

The automatic dispenser (1), as seen in FIG. 1, has buttons (not shown) that enable the set up of parameters that control the infusion volume and speed. The volume set up is quite simple and may be viewed on a crystal display (not shown).

There are at least three different speeds for piston displacement: slow, medium and fast, which may be chosen by using the buttons at the top of automatic dispenser (1) and may be viewed on the display. The suction speed of the liquid may be different from the dispensing speed; having the same dispensing speed variations. The volume to be injected may also be set up by the user, and the range of accuracy will vary with the volume of the reservoir (2) to be used.

Preferably, the liquid formulation is carried to the surgical center in a closed recipient, which is only opened in the surgical environment. The liquid formulation is then aspirated by the automatic dispenser (1). The automatic dispenser will preferably automatically recognize the total volume capacity of the connected reservoir. Once the liquid formulation is within the reservoir (2), it will no longer be in contact with the air. In another embodiment of the present invention, the reservoir (2) may be filled with the liquid formulation or liquid before being sent to the surgical setting, after its preparation.

The control of the number of infusions or injections is done by the automatic dispenser (1), which automatically calculates the total number of injections available once connected to the reservoir (2) and setting up the predetermined volume of each infusion. At each infusion made, the automatic dispenser (1) decreases the total number of available infusions, and this information is automatically shown on the display allowing the user to monitor the number of infusions. For example, if a reservoir (2) with a capacity of 25 ml is connected to the automatic dispenser (1) and the user sets up an infusion volume of 1 ml, the dispenser will automatically recognize that there are 25 infusions available. At each infusion made, this number is reduced on the display.

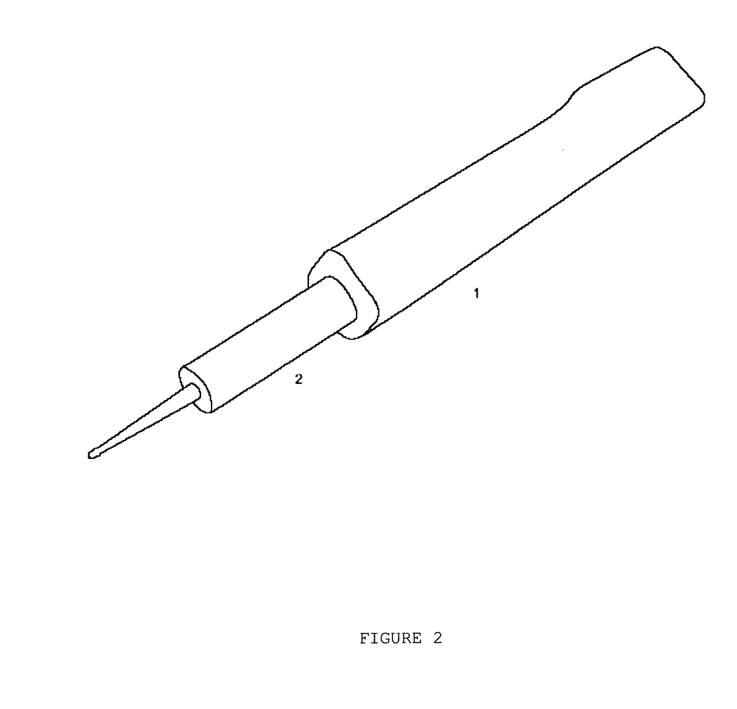

FIG. 2 shows a schematic drawing of the automatic dispenser (1) connected to the reservoir (2). The automatic dispenser (1) comprises a manual dosing device controlled by a microprocessor powered by batteries (not shown). The automatic dispenser (1) has electronic adjustments of the dispensed volumes, identifying the total doses available for the volume of the liquid aspired. The liquid aspired only comes into contact with the reservoir (2), having no contact with the electronic apparatus. The accuracy of the automatic dispenser (1) will vary with the volume of the reservoir (2) that is being used.

In a preferred embodiment, the reservoir (2) is sterile and packed individually. The sterile packaging containing the reservoir (2) is only opened in the surgical or outpatient center a few minutes before the infusion. The reservoir (2) is connected to the automatic dispenser (1). Subsequently, the dispenser (1) automatically recognizes the capacity of the reservoir (2) connected. Finally, the user presses a button in the dispenser (1) to aspire the liquid to be infused from its recipient to the reservoir (2).

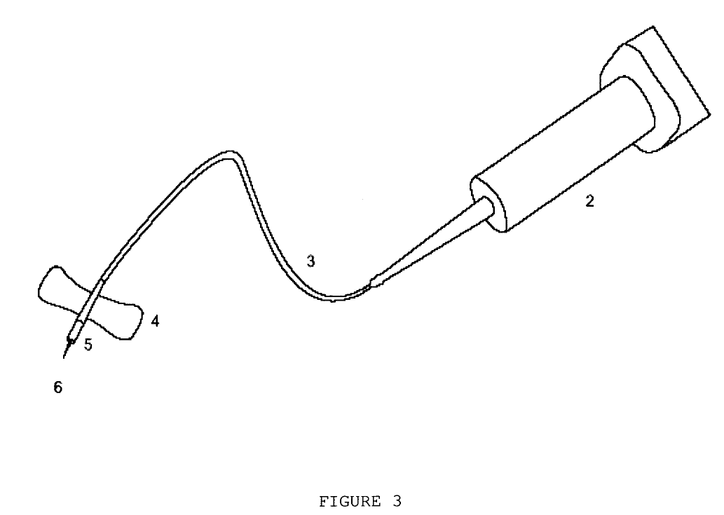

FIG. 3 shows the reservoir (2) connected by its bottom end to a duct (3). The duct (3) comprises in the opposite end a system consisting of a grip wing/handle (4) connected to a needle (6), comprising a depth insertion control device (5), limiting the insertion into a depth of 0, 1 cm to 1 cm, preferably a depth of 0, 5 cm (the infusion depth may vary according to the procedure characteristic).

In another embodiment of the present invention, the reservoir (2) connected by its bottom end to a duct (3) may be supplied as a refill filled in sterile conditions with the liquid to be infused (this filling may occur in the processing lab, immediately after the preparation of the liquid). In the surgical or outpatient center, at the moment of the surgical, clinical or minimally invasive cellular therapy procedure, the user simply needs to connect the filled reservoir to the automatic dispenser (1) and set up the parameters according to the desired type of infusion procedure and the device is ready to be used.

In a further embodiment of the present invention, a biocompatible mesh (not shown) may be used to assist the infusion procedure, showing the user the exact location of the infusion and determining the correct distances among the points of infusion.

In a further embodiment of the present invention, an image system (not shown) may be used to assist the user in taking images (pictures) of the infusion procedure, allowing a detailed report of the pre, per or post infusion procedure.

The infusion device of the present invention ensures higher safety, accuracy and simplicity handling in the infusion of cellular suspension or any kind of liquid comprising cells, directly to tissues and organs, during clinical, surgical or minimally invasive procedures.

Moreover, the infusion device of the present invention also ensures the control of the partial volumes and/or the total volumes injected, and may also assist the user in showing the number of infusions or injections available in the system. In addition, it ensures that the infusions or injections will be done in the specific points in the tissues or organs.

Furthermore, with the infusion device of the present invention, it is possible to ensure the traceability, reproducibility and robustness of medical, clinical and surgical procedures, which comprises steps of liquid formulations infusion in tissues or organs with the therapeutic or diagnostic purpose.

The reproducibility is guaranteed by the electronic system of the automatic dispenser (1) in which all the parameters used are determined by the pre-established procedures.

If necessary, adjustments may be made to the present invention. These include the addition of accessories; the addition of other substances to the duct and/or to the reservoir; the use of different materials to produce the various components of the device; the addition of RNA free components and/or any other adjustments that may be required depending on specific characteristics of the infusion procedure and/or the liquid formulation to be infused.

The infusion device of the present invention has advantages over the clinical or surgical infusion procedures commonly used (syringes), especially when the liquid infusion need to be injected directly in areas of difficult access of target tissues or organs. The device enables greater flexibility than a syringe, allowing the infusion in areas of difficult access. Moreover, the device ensures that the liquid to be infused will be administered in a simple way in a predetermined volume, by a simple press of a button.

Furthermore, the infusion device of the present invention allows the standardization of the procedure, since it guarantees the depth and the speed of the infusion, independently of who is handling the device. In addition, it ensures a faster medical procedure by enabling the user to work in a shorter period of time.

The present invention standardizes and ensures the accuracy, precision and reproducibility of the infusion, thereby increasing quality and providing better results in cell therapy procedures.

Although a preferred embodiment of the present invention is shown and described herein, it must be understood by those experienced in the related art that some modifications may be made without departing from the scope of this invention.

### REFERECES:

Tse, H-F; Siu, C-W; Zhu, S-G; Songyan, L; Zhang, Q-Y; Lai, W-H; Kwong, Y-L; Nicholls, J; Lau, C-P. Paracrine effects of direct intramyocardial implantation of bone marrow derived cells to enhance neovascularization in chronic ischaemic myocardium. European J Heart Failure 2007; 9: 747-53.

Steinhoff, G; Choi, Y-H; Stamm, C. Intramyocardial bone marrow stem cell treatment for myocardial regeneration. European Heart J Supplements 2006; 8(Sup. H): H32-H39.

Ting, A E; Mays, R W; Frey, M R; Hof, W V; Medicetty, S; Deans, R. Therapeutic pathways of adult stem cell repair. Critical Reviews Oncology/Hematology 2008; 65: 81-93.

Guan, K; Hasenfuss, G. Do stem cells in the heart truly differentiate into cardiomyocites ? J Molecular and Cellular Cardiology 2007; 43: 377-83.

Anversa, P; Leri, A; Rota, M; Hosoda, T; Bearzi, C; Urbanek, K; Kajstura, J; Bolli, R. Concise review: stem cells, myocardial regeneration, and methodological artifacts. Stem Cells 2007; 25: 589-601.

Haider, H Kh; Ashraf, M. Bone marrow stem cell transplantation for cardiac repair. Am J Physiol Heart Circ Physiol 2005; 288: 2557-67.

Laflamme, MA; Zbinden, S; Epstein, SE; Murry, CE. Cell-based therapy for myocardial ischemia and infarction: pathofhysiological mechanisms. Ann Rev Pathol Mech Dis 2007; 2: 307-39.

Losordo, D W; Renault, M-A. Therapeutic myocardial angiogenesis. Microvascular Research 2007; 74: 159-71.

Garbuzova-Davis, S., et al. Intravenous and Intraspinal Transplantation of Umbilical Cord Blood Cells in a Mouse Model of Amyotrophic Lateral Sclerosis. in Society for Neuroscience. 2002.

Garbuzova-Davis, S., et al., Intravenous administration of human umbilical cord blood cells in a mouse model of amyotrophic lateral sclerosis: distribution, migration, and differentiation. J Hematother Stem Cell Res, 2003. 12(3): p. 255-70.

Saporta, S., et al., Human umbilical cord blood stem cells infusion in spinal cord injury: engraftment and beneficial influence on behavior. J Hematother Stem Cell Res, 2003. 12(3): p. 271-8.

Garbuzova-Davis, S., et al. Compromised Blood-Brain Barrier in Early and Late Symptomatic ALS Mice. in Society for Neuroscience. 2005.

Garbuzova-Davis, S., et al. Dose-response study of human umbilical cord blood cells in treatment of ALS. in Society for Neuroscience. 2006.

Chen, J., et al., Intravenous administration of human umbilical cord blood reduces behavioral deficits after stroke in rats. Stroke, 2001. 32(11): p. 2682-8.

Sanchez-Ramos, J. R., et al., Expression of neural markers in human umbilical cord blood. Exp Neurol, 2001. 171(1): p. 109-15.

Zigova, T., et al., Human umbilical cord blood cells express neural antigens after transplantation into the developing rat brain. Cell Transplant, 2002. 11(3): p. 265-74.

Willing, A. E., et al., Intravenous versus intrastriatal cord blood administration in a rodent model of stroke. J Neurosci Res, 2003. 73(3): p. 296-307.

Henning, R. J., et al., Human umbilical cord blood mononuclear cells for the treatment of acute myocardial infarction. Cell Transplant, 2004. 13(7-8): p. 729-39.

Vendrame, M., et al., Infusion of human umbilical cord blood cells in a rat model of stroke dosedependently rescues behavioral deficits and reduces infarct volume. Stroke, 2004. 35(10): p. 2390-5.

Garbuzova-Davis, S., et al., Transplantation of human umbilical cord blood cells benefits an animal model of Sanfilippo syndrome type B. Stem Cells Dev, 2005. 14(4): p. 384-94.

Newman, M. B., et al., Stroke-induced migration of human umbilical cord blood cells: time course and cytokines. Stem Cells Dev, 2005. 14(5): p. 576-86.

Vendrame, M., et al., Anti-inflammatory effects of human cord blood cells in a rat model of stroke. Stem Cells Dev, 2005. 14(5): p. 595-604.

Newcomb, J. D., et al., Timing of cord blood treatment after experimental stroke determines therapeutic efficacy. Cell Transplant, 2006. 15(3): p. 213-23.

Newman, M. B., et al., Cytokines produced by cultured human umbilical cord blood (HUCB) cells: implications for brain repair. Exp Neurol, 2006. 199(1): p. 201-8.

Vendrame, M., et al., Cord blood rescues stroke-induced changes in splenocyte phenotype and function. Exp Neurol, 2006. 199(1): p. 191-200.

Li, Y. & Chopp, M. Marrow stromal cell transplantation in stroke and traumatic brain injury. Neurosci. Lett. 2009, 456(3), 120-3.

Orlic D, Kajstura J, Chimenti S, et al. Bone marrow cells regenerate infarcted myocardium. Nature 2001;410:701-5

Lunde K, Solheim S, Aakhus S, et al. Intracoronary injection of mononuclear bone marrow cells in acute myocardial infarction. N Engl J Med 2006;355:1199-209.

Schachinger V, Assmus B, Britten M B, et al. Transplantation of progenitor cells and regeneration enhancement in acute myocardial infarction: final one-year results of the TOPCARE-AMI trial. J Am Coll Cardiol 2004;44:1690-9.

Schachinger V, Erbs S, Elsasser A, et al. Intracoronary bone marrow derived progenitor cells in acute myocardial infarction. N Engl J Med 2006;355:1210-21.

Schachinger V, Erbs S, Elsasser A, et al. Improved clinical outcome after intracoronary administration of bone-marrow-derived progenitor cells in acute myocardial infarction: final 1-year results of the REPAIR-AMI trial. Eur Heart J 2006;27:2775-83.

Chen S L, Fang WW, Ye F, et al. Effect on left ventricular function of intracoronary transplantation of autologous bone marrow mesenchymal stem cell in patients with acute myocardial infarction. Am J Cardiol 2004;94:92-5.

Meyer G P, Wollert K C, Lotz J, et al. Intracoronary bone marrow cell transfer after myocardial infarction: eighteen months' follow-up data from the randomized, controlled BOOST (Bone Marrow Transfer to Enhance ST-Elevation Infarct Regeneration) trial. Circulation 2006; 113:1287-94.

Janssens S, Dubois C, Bogaert J, et al. Autologous bone marrowderived stem-cell transfer in patients with ST-segment elevation myocardial infarction: double-blind, randomised controlled trial. Lancet 2006;367:113-21.

Strauer B E, Brehm M, Zeus T, et al. Repair of infarcted myocardium by autologous intracoronary mononuclear bone marrow cell transplantation in humans. Circulation 2002;106:1913-8.

Bartunek J, Vanderheyden M, Vandekerckhove B, et al. Intracoronary injection of CD133-positive enriched bone marrow progenitor cells promotes cardiac recovery after recent myocardial infarction: feasibility and safety. Circulation 2005;112:1178-83.

Kang H J, Lee H Y, Na S H, et al. Differential effect of intracoronary infusion of mobilized peripheral blood stem cells by granulocyte colony-stimulating factor on left ventricular function and remodeling in patients with acute myocardial infarction versus old myocardial infarction: the MAGIC CELL-3-DES randomized, controlled trial. Circulation 2006;114:1145-51.

Li Z A, Zhang M, Jing Y Z, et al. The clinical study of autologous peripheral blood stem cell transplantation by intracoronory infusion in patients with acute myocardial infarction (AMI). Int J Cardiol 2007;115:52-6.

Assmus B, Schachinger V, Teupe C, et al. Transplantation of Progenitor Cells and Regeneration Enhancement in Acute Myocardial Infarction (TOPCARE-AMI). Circulation 2002;106:3009-17.

Ge J, Li Y, Qian J, et al. Efficacy of emergent transcatheter transplantation of stem cells for treatment of acute myocardial infarction (TCT-STAMI). Heart 2006;92:1764-7.

Meluzin J, Mayer J, Groch L, et al. Autologous transplantation of mononuclear bone marrow cells in patients with acute myocardial infarction: the effect of the dose of transplanted cells on myocardial function. Am Heart J 2006;152:975e9-15.

Ministério da Saúde, Governo do Brasil, 2005.

Beeres, S L M A; Bax, J J; Dibbets-Schneider, P; Stokkel, M P M; Fibbe, W E; van der Wall, E E; Schalij, M J. Sustained effect of autologous bone marrow mononuclear cell injection in patients with refractory angina pectoris and chronic myocardial ischemia: twelve-month follow-up results. Am Heart J 2006; 152: 684.e11-684.e16.

Losordo, D W; Schatz, R A; White, C J; Udelson, J E; Veereshwarayya, V; Durgin, M; Poh, K K; Weinstein, R; Kearney, M; Chaudhry, M; Burg, A; Eaton, L; Heyd, L; Thorne, T; Shturman, L; Hoffmeister, P; Story, K; Zak, V; Dowling, D; Traverse, J H; Olson, R E; Flanagan, J; Sodano, D; Murayama, T; Kawamoto, A; Kusano, K F; Wollins, J; Welt, F; Shah, P; Soukas, P; Asahara, T; Henry, T D. Intramyocardial transplantation of autologous CD34+ stem cells for intractable angina: A phase I/IIa double-blind, randomized controlled trial. Circulation 2007; 115: 3165-72.

van Ramshorst, J.; Bax, J. J.; Beeres, S. L. M. A.; Dibbets-Schneider, P.; Roes, S. D.; Stokkel, M. P. M; Roos, A.; Fibbe, W. E.; Zwaginga, J. J.; Boersma, E.; Schalij, M. J.; Atsma, D. E. Inramyocardial bone marrow cell injection for chronic myocardial ischemia. A randomized controlled trial. JAMA 2009; 301 (19): 1997-2004.

Perin, E C; Dohmann, H F R; Borojevic, R; Silva, S A; Sousa, A L S; Mesquita, C T; Rossi, M I D; Carvalho, A C; Dutra, H S; Silva, G V; Belem, L; Vivacqua, R; Rangel, F O D; Esporcatte, R; Geng, Y J; Vaughn, W K; Assad, J A R; Mesquita, E T, Willerson, J T. Transendocardial, autologous bone marrow cell transplantation for severe, chronic ischemic heart failure. Circulation 2003; 107: 2294-2302.

Fuchs, S; Satler, L F; Kornowski, R.; Okubagzi, P; Weisz, G; Baffour, R; Waksman, R.; Weissman, N J; Cerqueira, M; Leon, M B; Epstein, SE. Catheter-based autologous bone marrow myocardial injection in no-option patients with advanced coronary artery disease. A feasibility study. J. Am. Coll. Cardiol. 2003; 41: 1721-4.

Fuchs, S; Kornowski, R.; Weisz, G; Satler, L F; Smits, P C; Okubagzi, P; Baffour, R; Aggarwal, A; Weissman, N J; Cerqueira, M; Waksman, R.; Serrruys, P; Battler, A; Moses, J W; Leon, M B; Epstein, S E. Safety and feasibility of transendocardial autologous bone marrow cell transplantation in patients with advanced heart disease. Am J Cardiol 2006; 97: 823-9.

Tse, H-F; Thambar, S.; Kwong, Y-L; Rowlings, P; Bellamy, G; McCrohon, J; Bastian, B; Chan, J K F; Lo, G; Ho, C-L; Lau, C-P. Safety of catheter-based intramyocardial autologous bone marrow cells implantation for therapeutic angiogenesis. Am J Cardiol 2006; 98: 60-2.

Tse, H-F; Kwong, Y-L; Chan, J K F; Lo, G; Ho, C-L; Lau, C-P. Angiogenesis in ischaemic myocardium by intramyocardial autologous bone marrow mononuclear cells implantation. Lancet, 2003; 361: 47-49.

Tse, H-F; Thambar, S.; Kwong, Y-L; Rowlings, P; Bellamy, G; McCrohon, J; Bastian, B; Chan, J K F; Lo, G; Ho, C-L; Parker, A; Hauser, T H; Lau, C-P. Comparative evaluation of long-term clinical efficacy with catheter-based percutaneous intramyocardial autologous bone marrow cell implantation versus laser myocardial revascularization in patients with severe coronary artery disease. Am Heart J 2007; 154: 982.e1-982.e6.

Henning, R. J.; Burgos, J. D.; Vasko, M.; Alvarado, F.; Sanberg, C. D.; Sanberg, P. R.; Morgan, M. B. Human cord blood cells and myocardial infarction: effect of dose and route of administration on infarct size. Cell transpl. 2007, 16(9), 907-17.

Hossne, N. A. Jr.; Invitti, A. L; Buffolo, E.; Azevedo, S.; Oliveira, J. S. R.; Stolf, N. G; Cruz, L. E.; Sanberg, P. R. Refractory Angina Cell Therapy (ReACT) Involving Autologous Bone Marrow cells in Patients Without Left Ventricular Dysfunction: A Possible Role for Monocytes. (2009) Cell Transplantatio, v.18, n.12, pp.1299-1310.

1. Controlled infusion device for liquid formulations comprising cells, to be used in cellular therapeutic procedures, characterized in that it comprises an automatic dispenser (1) connected to a reservoir (2), which is connected to a duct (3), presenting in its opposite end a system consisting of a grip wing (4) connected to a needle (6) comprising a depth insertion control device (5).

2. Controlled infusion device for liquid formulations comprising cells, to be used in cellular therapeutic procedures, characterized in that it comprises an automatic dispenser (1) connected to a reservoir (2), which is directly connected to a needle (6).

3. Device, according to any one of claim 1 or 2, characterized in that the automatic dispenser (1) comprises an infusion volume control system, an aspiration and infusion speed control system and an infusion number control system.

4. Device, according to claim 3, characterized in that the automatic dispenser (1) comprises a system, which permits identifying the volume of the reservoir to which such system is connected and calculate the number of available infusions according to the infusion volume determined by the user.

5. Device, according to any one of claim 3 or 4, characterized in that the automatic dispenser (1) comprises a display for visualizing and monitoring the parameters defined in claim 3 or 4.

6. Device, according to any one of claims 1 to 5, characterized in that the reservoir (2) is made of plastic.

7. Device, according to any one of claims 1 to 5, characterized in that the duct (3) is made of a malleable material.

8. Device, according to claim 7, characterized in that the duct (3) is made of PVC.

9. Device, according to any one of claims 1 to 8, characterized in that the duct (3) has a length between 5 and 50 cm.

10. Device, according to any one of claims 1 to 8, characterized in that the needle (6) has a caliber between 0, 36 mm to 0, 91 mm.

11. Device, according to any one of claims 1 to 8, characterized in that the depth control device (5) allows the insertion of the needle (6) into a depth of 0, 1 cm to 1 cm.

12. Device, according to any one of claims 1 to 11, characterized in that it comprises an image system.

13. Device, according to claim 12, characterized in that the image system is a micro-camera.

14. Device, according to any one of claim 12 or 13, characterized in that the image system is connected to the grip wing (4).

15. Device, according to any one of claims 1 to 14, characterized in that it comprises a biocompatible mesh, which allows the exact location of the infusion.

16. Device, according to any one of claims 1 to 15, characterized in that it is used specifically for clinical or surgical infusion of liquid formulations comprising cells for the treatment of ischemic and non-ischemic diseases.
